(19) United States
(12) Patent Application Publication
(10) Pub. No.: US 2003/0163072 A1
(43) Pub. Date: Aug. 28, 2003

(54) Wound protector

(76) Inventor: Cristian Hueso, La Jolla, CA (US)

Correspondence Address: CRISTIAN HUESO, P.O. Box 301, La Jolla, CA, 92038, US

(21) Appl. No.: 10/170,099

(22) Filed: Mar. 3, 2003

Related U.S. Application Data
(60) Provisional application No. 60/359,797, filed on Feb. 26, 2002.

(51) Int. Cl. A61F 13/00

(52) U.S. Cl. 602/41

(57) ABSTRACT

The wound protector device designed with using the geometric conic arch in several different sizes. It is made of hard-flexible materials; attached to bands on its sides which are adherible to the skin, and gives a new and better physical protection to any superficial wound on the human body.

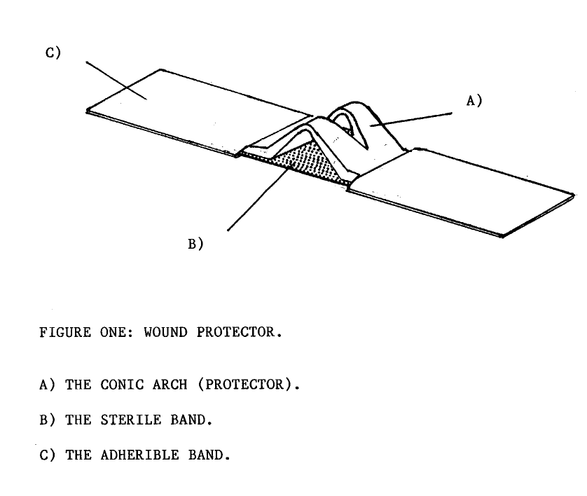

### CROSS-REFERENCE

NOT APPLICABLE.

### STATEMENT REGARDING FEDERALLY SPONSORED RESEARCH OR DEVELOPMENT

NOT APPLICABLE.

### REFERENCE TO A MICROFICHE APPENDIX

NOT APPLICABLE.

### BACKGROUND OF THE INVENTION

The wound protector pertains to the field of first aid and medical instruments, intended to protect superficial wounds on the human body.

### DESCRIPTION OF THE RELATED ART

The wound protector is a design developed to cover any superficial wound on the human body (especially the legs and arms) its porpuse is to protect the wound against any object that might strike it.

### BRIEF SUMMARY OF THE INVENTION

The advantages of the wound protector are especially to avoid pain and help the healing process with any superficial wound.

### BRIEF DESCRIPTION OF THE SEVERAL VIEWS OF THE DRAWING

The drawing includes one figure divided in three parts:

a) the conic arch (protector).

b) the sterile band.

c) the adherible band.

### DETAILED DESCRIPTION OF THE INVENTION.

The wound protector is designed to use on the human body as a protection for superficial wounds: it is held by bands that stick to the skin. The wound protector with its conic arch form will protect any superficial wound without touching it, protecting the wound against any object that might cause pain or cillateral damages.

The difference between the wound protector and regular first aid bandages is the “conic arch shaped protector” is made of hard_flexible materials with a new and extra protection that does more than just act a sterile band as do regular first aid bandages.

This conic arch protector is mede in many different shapes and sizes with air vents to allow ventilation, with several ways of attaching the sterile band under the conic arch with adherible bands on both sides.

What I claim as my invention is

1. The conic arch device that provides physical protection to any superficial wound on the human body; protecting against any object that might strike it and cause pain or prevent the healing process.
